(19) United States
(12) Patent Application Publication
(10) Pub. No.: US 2014/0348567 A1
(43) Pub. Date: Nov. 27, 2014

(54) APPARATUS FOR CARRYING AND DISPENSING CHALK

(71) Applicants: Daniel Allen Delisle, Belmont, CA (US); Brian Scott Steidle, Waterford, VA (US)

(72) Inventors: Daniel Allen Delisle, Belmont, CA (US); Brian Scott Steidle, Waterford, VA (US)

(21) Appl. No.: 13/898,595

(22) Filed: May 21, 2013

(51) Int. Cl. A45D 33/00; A61Q 15/00; A61K 8/02

(52) U.S. Cl. CPC A45D 33/005; A61K 8/0275; A61Q 15/00
USPC 401/200

(57) ABSTRACT

An apparatus for carrying and dispensing chalk for use by an athlete engaged in an activity such as gymnastics, weight lifting, rock climbing or other sports in which reduction of moisture from the palms of the hands and replacing it with friction to grip, a bar, dumbbell, rock, or other surface is essential to successfully execute and prevail in the activity. The chalk carrying and dispensing apparatus of the invention is lightweight, compact and capable of attaching to the body with straps, clips, or other means as well as attaching to clothing by adhesive or stitching means for easy access of the chalk with either hand of the athlete.

### FIELD OF THE INVENTION

The present invention is generally related to sporting goods. More particularly the invention relates to an apparatus for carrying and dispensing gym chalk.

### BACKGROUND OF THE INVENTION

Chalk is a generic name used to describe a range of compounds, predominantly, calcium carbonate found in sedimentary rocks, magnesium silicate in talcum powder, calcium sulfate (gypsum) used to form blackboard and sidewalk chalks, and magnesium carbonate commonly referred to as gym chalk applied to the hands to improve the grip of an athlete involved in sports such as, gymnastics, weight lifting and rock climbing to name a few. The primary benefit derived from the use of gym chalk in these sports is a reduction and elimination of moisture from the palms of the hands which in turn promotes the required friction between the skin on the palms of the hands and the surface with which it interacts, for example, the bars used in gymnastics, the weights in weight lifting, the rock surface in rock climbing, or the rope in a tug of war.

Traditionally, gym chalk in its various forms such as chunks, powder, or liquid are stored in bags or other receptacles and either left on the floor of a gym with easy access by the athlete during their workout or the bags or receptacles carried by the athletes on their bodies by some fastening means such as a lanyard or waist belt. Rock climbers typically fasten a bag filled with chalk around their waists while climbing, allowing the climber to access the chalk with their hands during a climb. Gymnasts and weight lifters do not typically carry a bag of chalk on their bodies during their activities, because the added weight and awkwardness of an ancillary appendage may be a burden to their activity. Therefore, gymnasts and weight lifters generally have a bag filled with chalk adjacent to their activity station.

In general, the chalk bags in the prior art are difficult to access during an activity. For example, chalk bags designed for use by rock-climbers are generally hung around the waist of a climber with the bag held on the back side of the climber in order to keep it out of the way of the rock climbing surface and the climber's gear while the climber is trying to navigate up the rocks with their front sides closely hugging the rock surface. This positioning of the chalk bag on the back of a rock climber combined with the task of reaching back with one hand and trying to open the drawstring or flap type closed tops of the bags to access the chalk, is a maneuver if not executed correctly, can be dangerous, posing a risk to the climber of inadvertently losing their sole grip on the rock with their other hand. In addition, when the hand of the user is extracted from the chalk bag, a great deal of the lose chalk can spill out leading to chalk waste.

The chalk bags in the prior art that focus on use by gymnasts, weight lifters, javelin throwers and similar sports that require the removal of moisture from the palms of the hands and create friction between the palm and the holding surface are generally large in size and cumbersome to carry around when the athlete is engaged in the sport.

Other deficiencies and limitations in the prior art chalk bags involve, ineffective lids and closures that allow chalk to spill easily out of the bags during activities, accumulation of chalk at the bottom of the bag or container, uneven distribution of the chalk within the bag or container, and the lack of a surface on the bags to dry the hands during an activity.

The present invention overcomes the deficiencies and limitations of chalk bags in the prior art by providing a chalk carrying apparatus that holds chalk without spillage and exposure to the elements; has the ability to distribute the chalk evenly throughout the bag; has a surface made of material that quickly absorbs the moisture/sweat from the palms of the hands during activities; and is compact, lightweight, and easily portable.

### SUMMARY OF THE INVENTION

The present invention is an improvement over prior art chalk bags and chalk holding containers and aims to provide a modified apparatus to the conventional chalk bags used in gyms by gymnasts, weight lifters and other athletes to get rid of moisture from the palms of their hands to create a frictional surface for grasping the bar, dumbbell, or surfaces of other devices.

The primary object of the present invention is to provide a chalk holding container with a substantially easier method of chalk distribution and prompt access to the chalk during an activity. Accordingly, the internal cavity of the apparatus is constructed with materials that allow the chalk to remain consistently distributed within the inside walls of the container without caking and settling to the bottom of the container or bag as is commonly experienced with the majority of chalk bags and containers known in the prior art.

In an exemplary embodiment of the present invention, the improved chalk bag is an apparatus for carrying and dispensing chalk comprising, an interior cavity for holding chalk, with a re-closeable opening at the top to provide access to the chalk held in the interior cavity. In some embodiments of the invention, the interior cavity of the apparatus is constructed of a moisture-wicking material to prevent moisture accumulation inside the bag which can result in caking of the chalk. In other embodiments of the invention, the interior cavity of the apparatus may be constructed from material that is not moisture-wicking.

In another embodiment of the present invention, the interior cavity of the apparatus may be lined with a material that has restricted porosity, such as polyethylene plastic sheeting or iron-on vinyl, to limit the passage of chalk to the outside of the container resulting in loss of chalk. In a related embodiment of the invention, the outermost side of the apparatus may be constructed of a material with restricted porosity, such as polyethylene plastic sheeting or iron-on vinyl, to limit the passage of chalk from the inside cavity to the outside surface of the bag.

In yet another embodiment of the chalk carrying apparatus of the present invention, the interior cavity of the apparatus is lined with a porous, absorbent, material capable of holding chalk on the innermost side of the bag as well as permitting its passage to the outside surfaces, thus distributing chalk on both the innermost side and the outermost sides of the bag for access to chalk from both sides of the bag.

In yet another embodiment of the invention, one of the sides of the interior cavity of the apparatus may be constructed of a material with restricted porosity, such as polyethylene plastic sheeting or iron-on vinyl, to limit the passage of chalk from the inside cavity to the outside surface of the bag, while the other side of the interior cavity of the apparatus is lined with a porous, absorbent, material capable of holding chalk on the innermost side of the bag as well as permitting its passage to the outside surface, thus distributing chalk on only one side of the bag. In another embodiment of the present invention, one of the outer surfaces of the chalk bag may be constructed of a material with restricted porosity, while the other outer surface of the apparatus is a porous, absorbent, material capable of holding chalk as well as permitting its passage to the outside surface.

Another object of the present invention is to provide a chalk carrying apparatus that facilitates faster and more efficient drying of the hands using an outside surface that wicks moisture from the palms of the hands. Accordingly, in another embodiment of the present invention, the outer surface of the chalk bag or container may be lined with a pile-type textured material to allow for the abstraction of accumulated chalk and moisture from the palms of the hands of the user.

Another object of the present invention is to provide a chalk holding bag or container that can be attached to the body of the user for easy access of the chalk. Accordingly, the chalk carrying apparatus of the present invention is constructed with the capability of attachment to clothing worn by a user or strapped around the user's hips, legs, waist, or hand.

Yet another object of the present invention is to provide a chalk holding bag or container that is lightweight and compact enough to be easily transportable by gymnasts and other athletes as well as rock climbing enthusiasts.

In this summary of the invention and in the specification in general, the various references to an “exemplary embodiment,” “another embodiment,” “alternate embodiment,” and/or “yet another embodiment” do not necessarily refer to the same embodiment (s). Rather, these references to the various embodiments generally mean that a particular feature, structure, or characteristic described in conjunction with a particular embodiment is included in at least some embodiments, if not all embodiments and therefore should not be construed to limit the scope of the invention. It is to be understood by those skilled in the art, that the invention can be implemented in embodiments other than those recited in this summary of the invention.

The features and advantages of the present invention as described in this summary of the invention will be best understood and become obvious to a person skilled in the art when viewed in conjunction with the detailed description of the invention accompanied by the drawings and the claims that follow.

### BRIEF DESCRIPTION OF THE DRAWINGS

FIG. 1 is a perspective view of the front side of one embodiment of the chalk carrying apparatus of the present invention.

FIG. 2 is a perspective view of the back side of one embodiment of the chalk carrying apparatus of the present invention.

FIG. 3 is a perspective view of the back side of an alternate embodiment of the chalk carrying apparatus of the present invention.

FIG. 4 is a perspective view of the back side of yet another embodiment of the chalk carrying apparatus of the present invention.

FIG. 5 is a perspective view of the back side of yet another embodiment of the chalk carrying an apparatus of the present invention.

FIG. 6 is a perspective view of the top opening end of an embodiment of the chalk carrying apparatus of the present invention.

FIG. 7 is a perspective view of an example of the type of stitching employed in securing the sides and bottom end of the chalk carrying apparatus of the present invention.

FIG. 8 is an exploded perspective view of the panels of the different materials used to construct the chalk carrying apparatus of the present invention.

FIG. 9 is a perspective view of an embodiment of the chalk carrying apparatus of the present invention and its attachment to the body of the user.

FIG. 10 is a perspective view of the manner in which another embodiment of the chalk carrying apparatus of the present invention is attached to the body of the user.

FIG. 11 is a perspective view of yet another manner in which one embodiment of the chalk carrying apparatus of the present invention is strapped to the leg of the user.

FIG. 12 is a perspective view of the chalk carrying apparatus attached to the clothing of the user.

FIG. 13 is a perspective view showing a user filling one embodiment of the chalk carrying apparatus of the present invention with chalk.

FIG. 14 is a perspective view showing a user squeezing and massaging the chalk from the exterior of one embodiment of the chalk carrying apparatus of the present invention to evenly distribute the chalk contained in the interior cavity of the apparatus.

FIG. 15 is a perspective view of yet another manner in which one embodiment of the chalk carrying apparatus of the present invention is strapped to the leg of a user.

FIG. 16 is a perspective view of a user accessing the chalk from the interior cavity of the chalk carrying apparatus by palming or slapping the front surface of the apparatus, to get chalk on the palms of the hands, and pressing the palms of the hands against the front surface of the apparatus.

### DETAILED DESCRIPTION OF THE INVENTION

The present invention is an improved chalk carrying apparatus for efficiently dispensing chalk into the hands of athletes, rock climbers and other sports enthusiasts for whom elimination of moisture from the palms of their hand and creating a frictional surface to provide better grip is critical for safe and effective handling of surfaces for the successful execution of their sports.

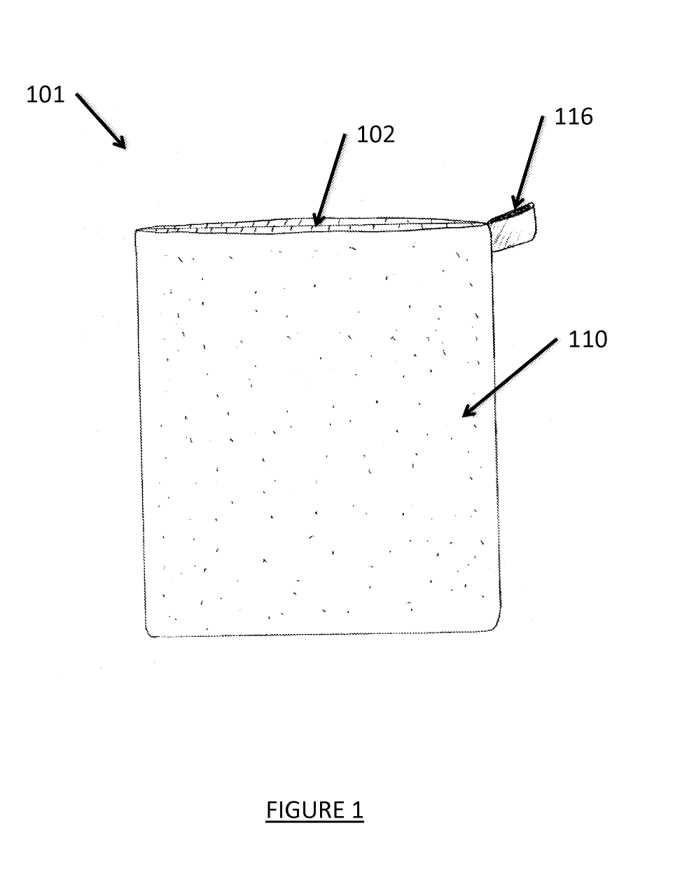

Referring now to the figures, more particularly, to FIG. 1 a perspective view of the front side of one embodiment of the chalk carrying apparatus 101 is shown. In this embodiment, the chalk carrying apparatus 101 is rectangular in shape with a width, preferably in the range of, between 3 inches to 6.5 inches and a height, preferably in the range of, between 4 inches to 8 inches. However, the size and shape may vary depending on the preferred substrate and location on the user's clothing or gear to which the chalk carrying apparatus 101 is attached and the orientation of the chalk carrying apparatus 101 may vary depending on the user's preferred configuration for attachment of the apparatus to their clothing. Furthermore, the size and shape of the bag may be bigger than the preferred range to accommodate additional chalk. In this view of the chalk carrying apparatus 101 the top re-closable opening, 102 can also be seen. This opening 102 opens to an interior cavity that is used to hold the chalk. The perspective side view of this embodiment of the chalk carrying apparatus 101 also shows a loop structure 116 to thread straps or cords for attachment to the body of the user using a carabineer, hook or other devices. The loop 116 is secured to the chalk carrying apparatus 101 using heavy nylon thread or other strong attachment means. A material 110 with moisture-wicking properties is used to construct the front side of the chalk carrying apparatus 101 to enable the user to use the surface to remove moisture from the palms of their hands. In another embodiment of the chalk carrying apparatus 101 of the present invention, the material 110 may have a texture to allow passage and accumulation of chalk as well as remove moisture from the palms of the hand. In general, the chalk carrying apparatus 101 may be constructed of material including, but not limited to, heavy duty cotton webbing, nylon, canvas, fleece and others that are durable for both indoor and outdoor use.

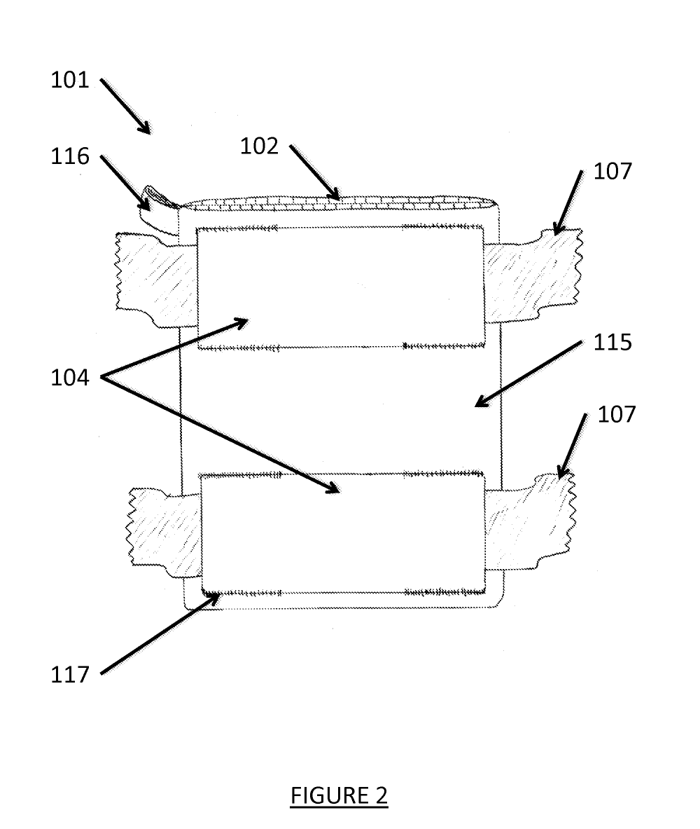

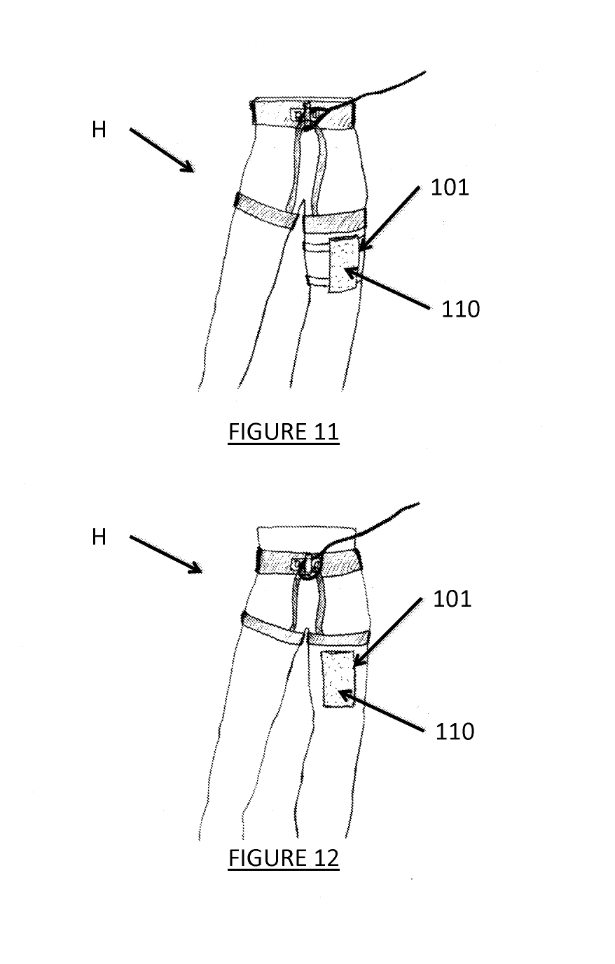

FIG. 2 is a perspective view of the back side of one embodiment of the chalk carrying apparatus 101 of the present invention showing straps 107 that are slid through strap loop structures 104 attached to the back side material 115 through attachment points 117 and secured using heavy nylon thread or other strong attachment means. In this embodiment of the chalk carrying apparatus 101 of the invention, two straps 107 are attached, one at the top and the other at the bottom of the back side material 115 so that they are oriented parallel to one another. The straps 107 typically range in length between 1 foot and 2.5 feet long, with a width between 0.5 inches and 3 inches. In this embodiment, each strap 107 may have fittings at the ends, such as buckles, hook-and-loop fastenings or other similar devices to secure the chalk carrying apparatus 101 to the user. The straps 107 may be used with fittings that enable the user to lengthen and shorten the straps 107 to more appropriately attach to the user. The straps 107 holding the chalk carrying apparatus 101 are generally fastened tightly around the user's waist, legs, or arms at a point where the user can easily access the chalk in the apparatus as shown in FIG. 11. A variety of fittings and fastenings types may be used without departing from the scope of the invention. In some embodiments of the invention, fittings and fastenings may not be used at all without departing from the scope of the invention. In those embodiments, the chalk carrying apparatus may be constructed to be set on the floor of the gym for use by the athlete

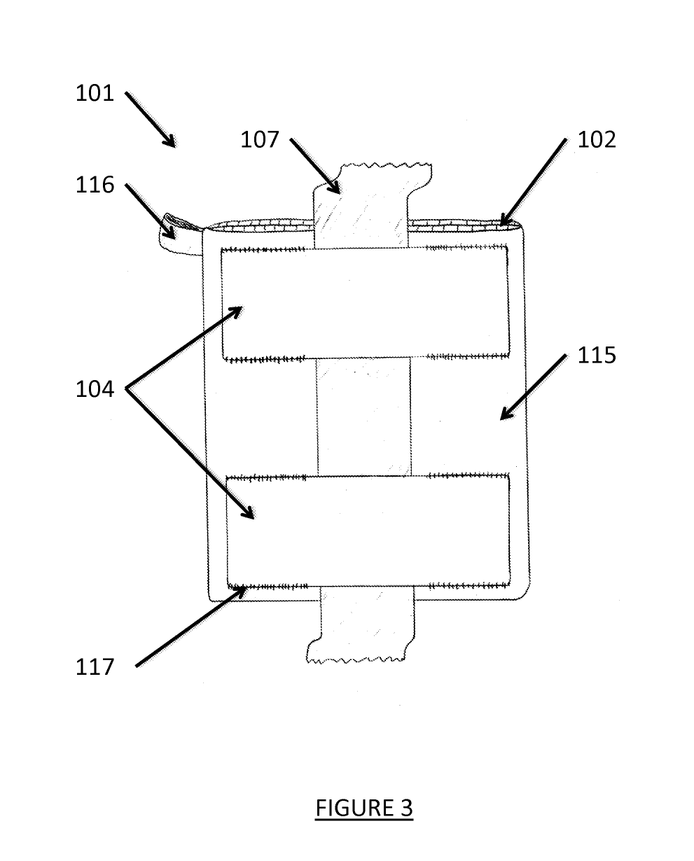

FIG. 3 is a perspective view of the back side of an alternate embodiment of the chalk carrying apparatus 101 of the present invention. In this embodiment of the invention, only one strap 107 is used to secure the chalk carrying apparatus 101 to the user. In this embodiment, just as in the embodiment described in FIG. 2 the strap 107 may have fittings at the ends, such as buckles, hook-and-loop fastenings or other similar devices to secure the chalk carrying apparatus 101 to the user. The strap 107 may be used with fittings that enable the user to lengthen and shorten the strap 107 to more appropriately attach to the user. This embodiment may be used in substantially the same way as shown in FIG. 11, but may also be used with a different strap 107 configuration, where the straps 107 may be used to attach to another article of clothing or gear, such as the leg-loop of a rock climbing harness.

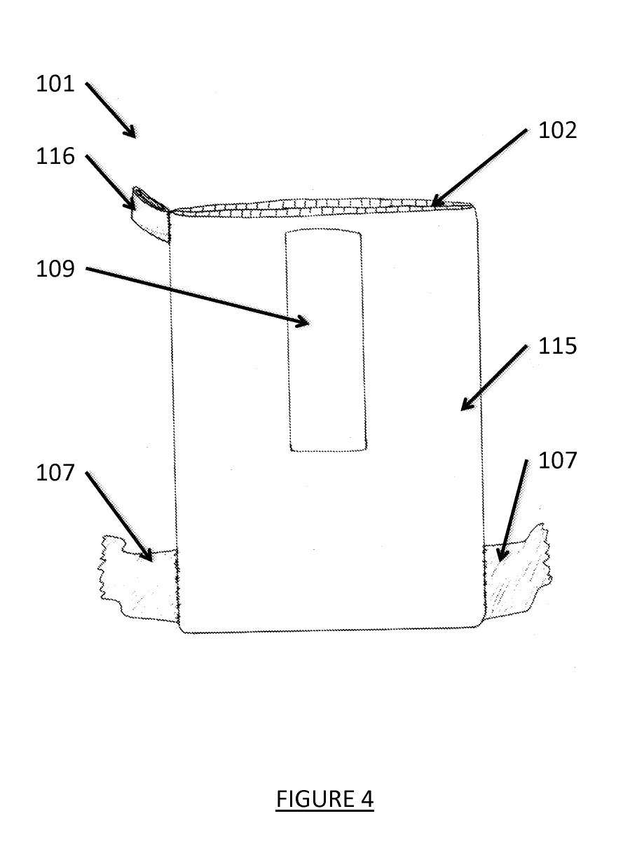

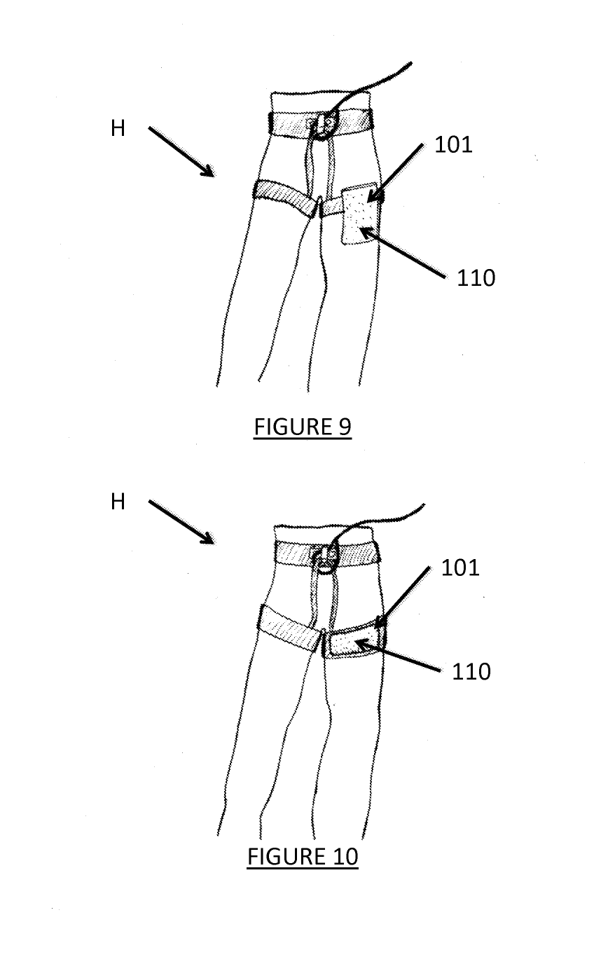

FIG. 4 shows an alternate embodiment of the chalk carrying apparatus 101 in which an attachment clip 109 may be used to attach to another article of clothing or gear, such as the leg-loop of a rock climbing harness, as shown in FIG. 9. The attachment clip 109 may be any material, size, or shape, and is intended to provide an alternate means of attaching the chalk carrying apparatus 101. Similar to the straps 107 of FIG. 1 and FIG. 2 and in addition to the attachment clip 109, the chalk carrying apparatus 101 may also include another strap 107, which may have fittings at the ends, such as buckles, hook-and-loop fastenings or other similar devices to secure the chalk carrying apparatus 101 to the user. This embodiment may be used in substantially the same way as described in FIG. 1 and FIG. 2, but may also be used with a different attachment configuration, (e.g., using the attachment clip 109).

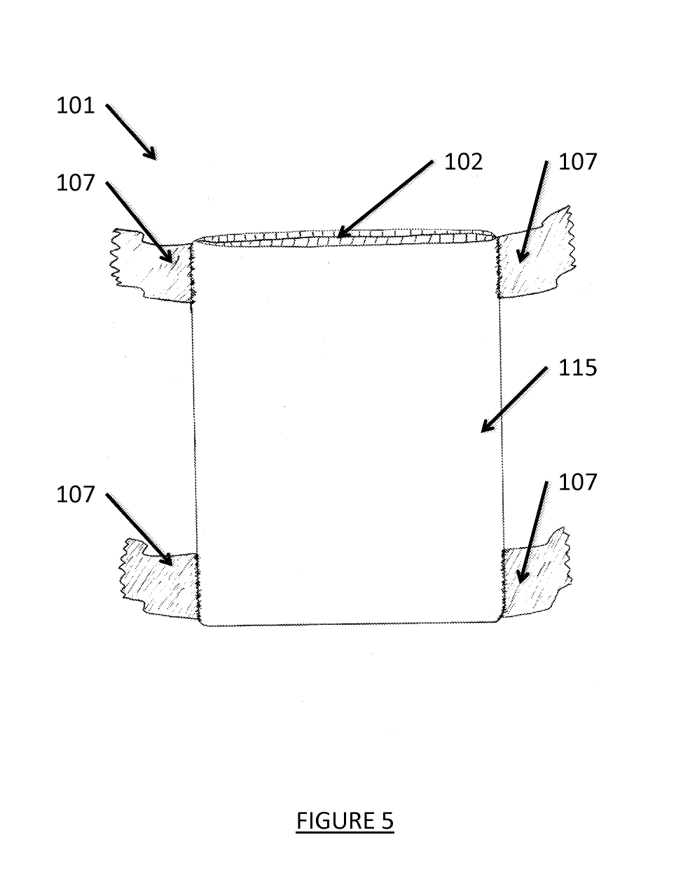

FIG. 5 is a perspective view of the back side of yet another embodiment of the chalk carrying apparatus 101 of the present invention. In this embodiment of the invention, a different arrangement of the straps 107 can be seen as they are attached to the back material 115. In this embodiment, the straps 107 may be attached to the side of the chalk carrying apparatus 101 with two at the top and two at the bottom of the apparatus at their attachment points, and may be typically secured with heavy nylon thread, or by other means for, strength. As in the previous embodiments shown in FIGS. 1, 2, 3, 4 each strap 107 may have fittings at the ends, such as buckles, hook-and-loop fastenings, or other similar devices to secure the chalk carrying apparatus 101 to the user.

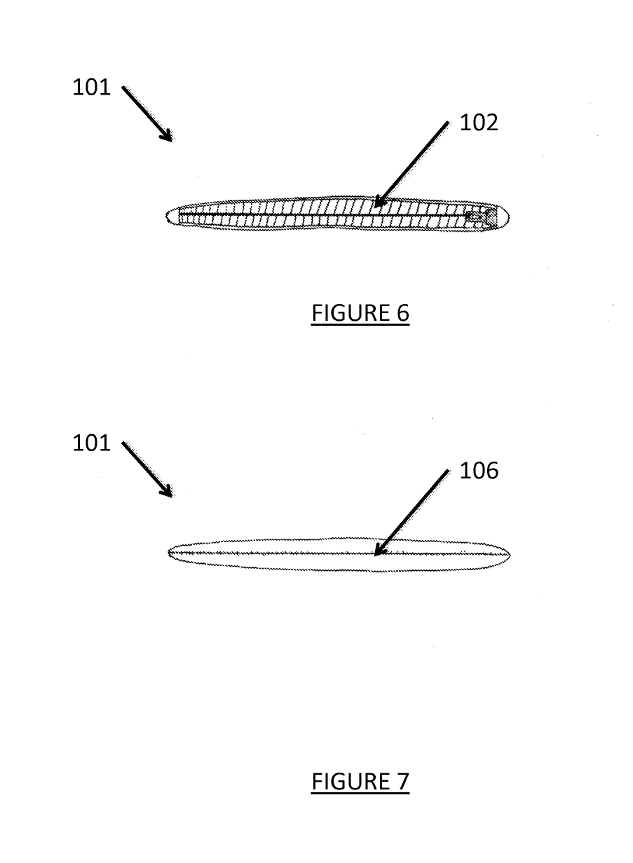

FIG. 6 is a perspective view of the top opening end of the preferred embodiment of the chalk carrying apparatus 101 of the present invention. In this view, the re-closable opening 102 at the top of the apparatus has a slider-operated zipper assembly, typically comprising a plastic zipper having two interlocking profiles and a slider 105 for opening and closing the zipper. The re-closable opening 102 thus limits chalk from escaping the chalk carrying apparatus 101 and may include additional reinforcement materials at the top corners 108 to prevent the chalk from spilling out of those corners when the apparatus is in use and when not in use. In other embodiments of the chalk carrying apparatus 101 non-zipper assembly related closures may be used to keep the opening 102 closed and secure from chalk spillage.

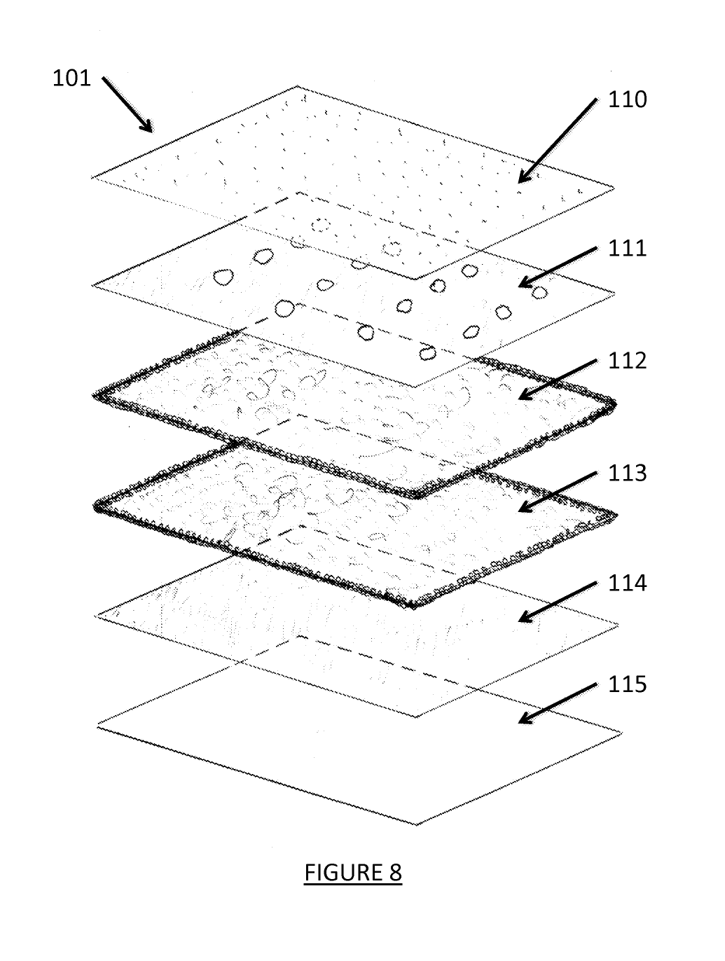

FIG. 7 is a perspective view of an example of the type of stitching employed in securing the sides and bottom end of the chalk carrying apparatus of the present invention. In this example, the bottom and side edges 106 of the chalk carrying apparatus 101 may be secured with stitching or other means of fastening to seal the individual material panels shown in FIG. 8 together and along with the zipper assembly at the top re-closable opening 102 helps in maintaining the integrity of the chalk carrying apparatus 101 in its ability to hold chalk without spillage.

FIG. 8 is an exploded perspective view of the panels of the different materials used to construct the chalk carrying apparatus 101 of the present invention. The main body of the chalk carrying apparatus 101 generally includes six panels, as shown in this figure: A front material 110 as seen in FIG. 1, A perforated panel 111 inboard and adjacent to the front material 110, two loft panels, 112 and 113 separated by a void, a non-perforated panel 114 inboard and adjacent to the back material 115 and the back material 115 as seen in FIGS. 2, 3, 4, and 5. For the purposes of this specification, panels are defined as a layer of different material; for instance, urethane-coated nylon would constitute as two panels, the urethane coating and the nylon, respectively.

By way of further explanation and clarification of the relations between and functions of these various panels, the front material 110 of the chalk carrying apparatus 101 may include material with properties that allow the passage of chalk and may include a material with moisture-wicking properties, to provide additional mitigation of unwanted moisture from the hands. The front material 110 may also include a material with pile type texture, to allow additional accumulation of chalk and provide additional abstraction of moisture from the hands. The perforated panel 111 inboard and adjacent to the front material 110 limits the amount of chalk passing through to the front material 110. The perforated panel 111 material may generally consist of a non-porous material with holes or openings to provide a limited amount of chalk to filter through to the front material 110. The number and size of the holes in the perforated panel 111 may vary depending on the amount of chalk preferred to be released to the front material 110. The two loft panels 112 and 113 preferably comprise an open-cell structure (e.g., sponge) or multi-component fibers in which the fiber cross-sections are arranged in the form of a matrix (e.g., batting) to hold and store chalk for distribution through the perforated panel 111 and front material 110. The void between the two batting layers, 112 and 113 provides an interior cavity for holding the chalk and providing its distribution through the perforated panel 111 and front material 110. The interior cavity and the two loft panels 112 and 113 of the chalk carrying apparatus 101 are designed such that chalk remains consistently distributed in all orientations, and in particular, in a vertically-oriented disposition. The amount of chalk distributed through the perforated panel 111 and front material 110 will be determined by the strength, frequency, and coverage area of the user's hand when it palms or slaps on the outside surface of the front material 110 of the chalk carrying apparatus 101. The non-perforated panel 114 inboard and adjacent to the back material 115 includes material with properties that limit the passage of chalk. The non-perforated panel 114 may include a less porous material, such as polyethylene plastic sheeting, to limit the passage of chalk to the back material 115. The back material 115 of the chalk carrying apparatus 101 may include moisture-wicking material, to limit moisture accumulation on the substrate/clothing worn by the user, the skin, or gear elements. The preferred panels may be made of any material that provides properties and performance as detailed above. Additional panels may be added, some panels replaced with other panels or, some panels removed without departing from the spirit and scope of the invention.

FIG. 9 is a perspective view of an alternate embodiment of the chalk carrying apparatus 101 of the present invention and its attachment to the body of the user. In this embodiment, an attachment clip 109 (not shown) may be used to attach to another article of clothing or gear, such as the leg-loop of a rock climbing harness on user H, with the outside surface of the front material 110 of the chalk carrying apparatus 101 facing away from the user H.

FIG. 10 is a perspective view of the manner in which another embodiment of the chalk carrying apparatus 101 of the present invention is attached to the body of the user. The figure shows the apparatus 101 affixed to the leg-loop of a rock climbing harness on a user H, with the outside surface of the front material 110 of the chalk carrying apparatus 101 facing away from the user H.

FIG. 11 is a perspective view of yet another manner in which one embodiment of the chalk carrying apparatus 101 of the present invention is strapped to the leg of a user H, with the outside surface of the front material 110 of the chalk carrying apparatus 101 facing away from the user H.

FIG. 12 is a perspective view of the chalk carrying apparatus 101 attached to the clothing, for example, a pair of pants of the user H, with the outside surface of the front material 110 of the chalk carrying apparatus 101 facing away from the user H.

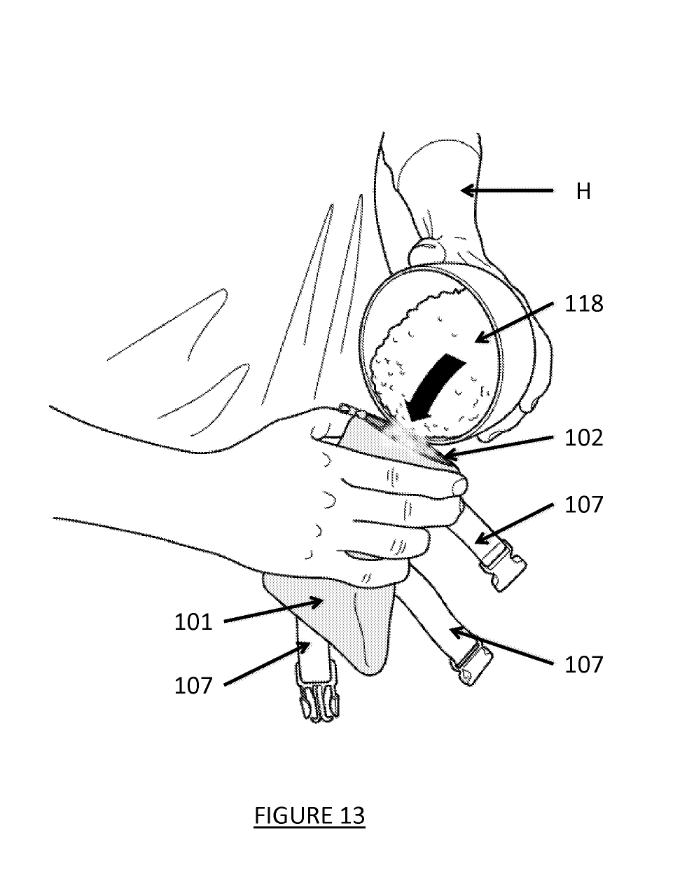

FIG. 13 is a perspective view showing a user H filling one embodiment of the chalk carrying apparatus 101 with loose chalk 118 through the top re-closable opening 102. The straps 107 hold the chalk carrying apparatus 101 against the body of the user.

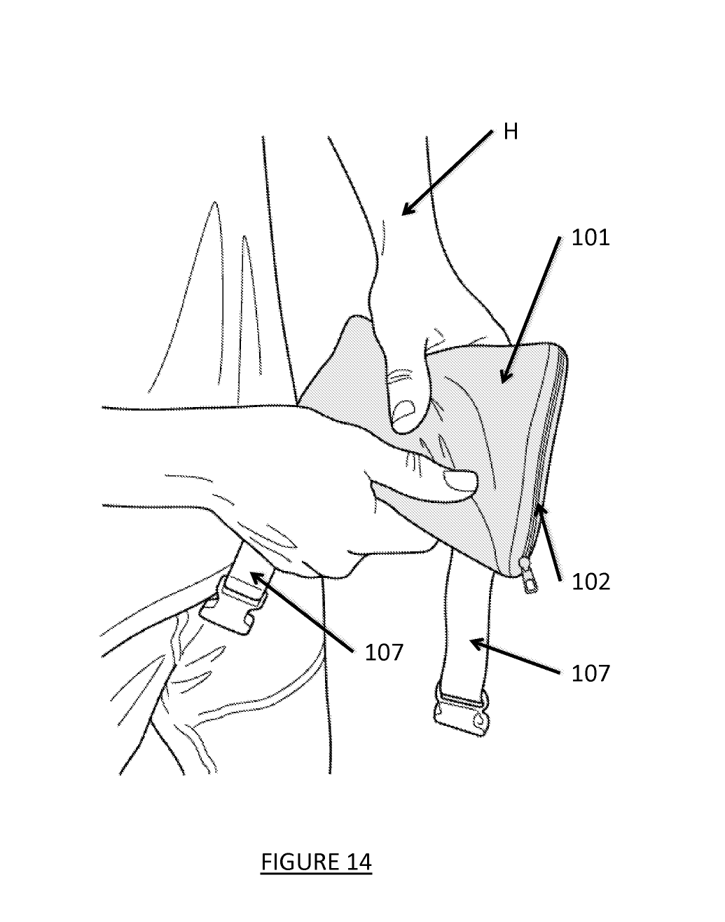

FIG. 14 is a perspective view showing a user H squeezing and massaging the chalk from the exterior of one embodiment of the chalk carrying apparatus 101 of the present invention to evenly distribute the chalk contained in the interior cavity of the apparatus. In this view, the opening 102 is closed with a slider-operated zipper assembly. In some embodiments, non-zipper related means such as hook and loop tape, snaps, or fold-over type arrangement may be employed to close the mouth of the chalk carrying apparatus.

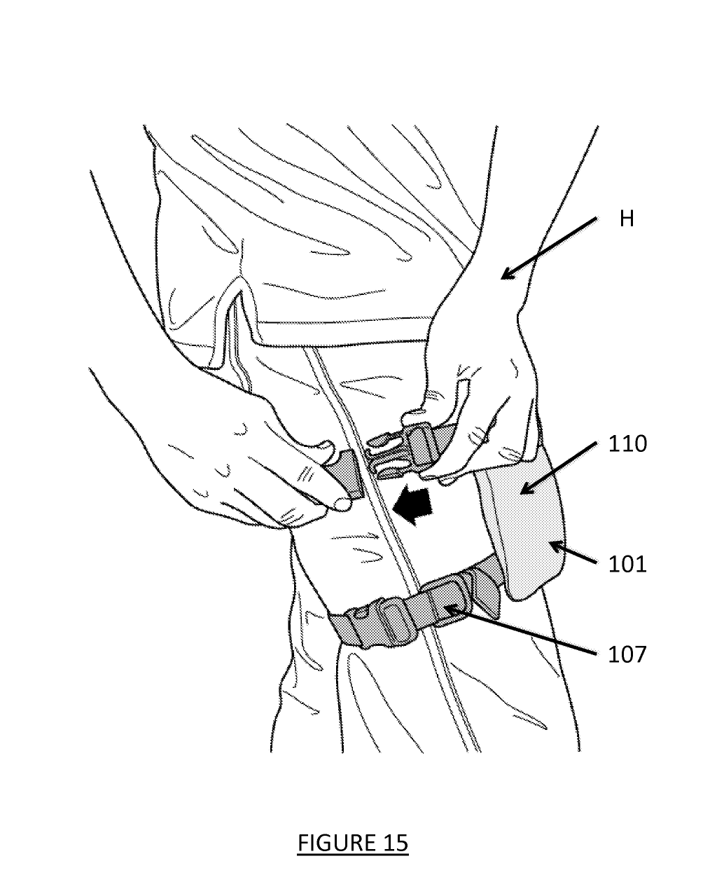

FIG. 15 is a perspective view of yet another manner in which one embodiment of the chalk carrying apparatus 101 of the present invention is strapped to the leg of the user H using straps 107.

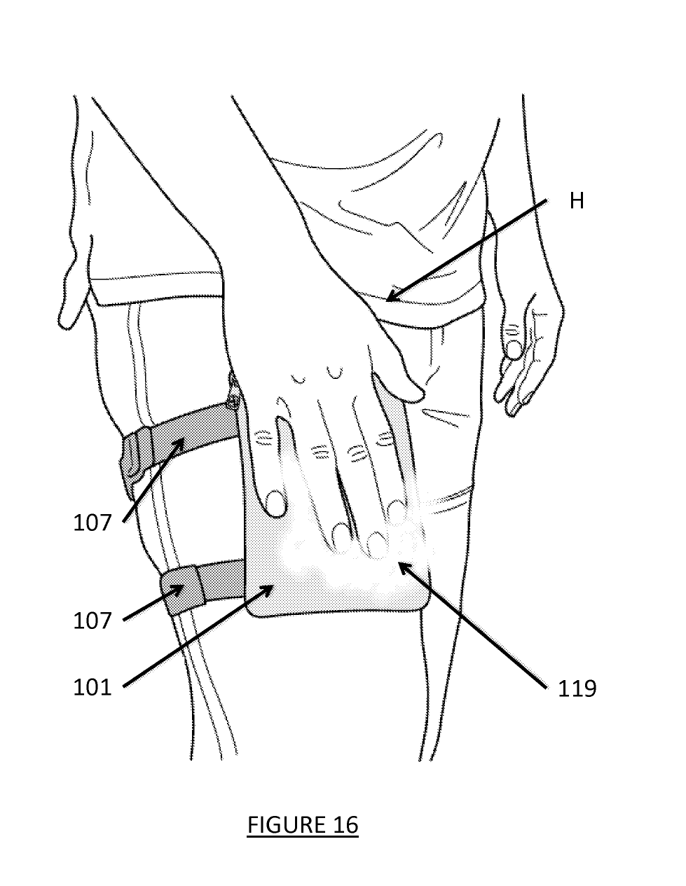

FIG. 16 is a perspective view of a user H accessing the chalk from the interior cavity of the chalk carrying apparatus 101 by palming or slapping the front surface of the apparatus, to get chalk on the palms of the hands 119, and pressing the palms of the hands against the front material 110 of the apparatus.

As depicted above in FIGS. 2, 3, 4, and 5 the chalk carrying apparatus 101 may have attachment methods, such as the straps 107, that may be configured in many different forms, such as with one strap 107 or multiple straps 107, an attachment clip 109, and may include straps 107 which are fixed or removable for more convenient storage or providing supplementary replacement parts. The chalk carrying apparatus 101 may also be affixed to clothing and gear by many different means. For example, FIG. 10 shows the chalk carrying apparatus 101 affixed to the leg-loop of a rock climbing harness and FIG. 12 shows the apparatus 101 affixed to an article of clothing such as a user's pants. The attachment method may include, but is not be limited to, stitching the chalk carrying apparatus 101 directly to the article of clothing or particular gear element, adhering the chalk carrying apparatus 101 with the use of adhering elements that do not require any stitching to support the weight and contents of the chalk carrying apparatus 101, while also providing adequate securement of the apparatus to the body of the user during sports activities. In some embodiments, iron-on adhesive prior arts may be used to securely and permanently affix the chalk carrying apparatus 101 to a garment surface or gear element once applied. Several other attachment methods are possible using methods not addressed above, along with variations of the given attachment methods as described. Therefore, any combination of the above-described attachment elements in all possible variations thereof, and other possible attachment methods not described, are encompassed by the invention unless otherwise indicated herein or otherwise clearly contradicted by context. It will be apparent to the skilled artisan that there are numerous changes that may be made in embodiments described herein without departing from the scope and spirit of the present invention, including, but not limited to, external pouches and re-closeable pockets integrated with the various panels, for carrying additional items.

While the principles of the invention have been described above in connection with specific novel embodiments, it is to be understood that these descriptions are made only by way of examples, and should not be construed as limitations on the scope of the invention. It should be understood and obvious to one skilled in the art that various modifications, including possible omissions, substitutions, and changes in the specifications of the chalk carrying apparatus are within the scope of the invention. For example, the various dimensions of the chalk carrying apparatus described herein may differ in other embodiments of the invention. As another example, the method of using the chalk carrying apparatus may differ, such as, but not limited to, using the apparatus with another piece of equipment, such as an article of clothing, or attaching or fixing to another part of the body such as an arm during an activity. In general, it should be understood and obvious to one skilled in the art that alternatives, modifications and variations of the embodiments of the invention may be construed as being within the spirit and scope of the appended claims.

What is claimed is:

1. An apparatus for carrying and dispensing chalk comprising:
a) an exterior front structure with a surface having properties allowing the passage of chalk;
b) an exterior back structure with a surface having properties preventing the passage of chalk;
c) an interior cavity structure having properties allowing the retention and distribution of chalk;
d) a re-closable opening structure to access the chalk held in the interior cavity of the apparatus; and
e) an attachment means to secure the apparatus to the body.

2. The apparatus as in claim 1 wherein the front structure, back structure, and the interior cavity structures comprise in total at least six panels.

3. The apparatus as in claim 2 wherein the panels of the front structure and the back structure are constructed from materials having moisture-wicking properties.

4. The apparatus as in claim 2 wherein the panels of the front structure and the back structure are constructed from materials that do not have moisture-wicking properties.

5. The apparatus as in claim 2 wherein the panels of the interior cavity structure are constructed from materials that have moisture-wicking properties.

6. The apparatus as in claim 2 wherein the panels of the interior cavity structure are constructed from materials that do not have moisture-wicking properties.

7. The apparatus as in claim 1 wherein the exterior front structure having properties allowing the passage of chalk is constructed from one panel made of a porous material with moisture-wicking properties and a second panel made of a material with perforations to allow for the passage of chalk from the interior cavity.

8. The apparatus as in claim 1 wherein the exterior front structure having properties preventing the passage of chalk is constructed from one panel made of a non-porous material with moisture-wicking properties and a second panel made of a material with no perforations.

9. The apparatus as in claim 1 wherein the exterior back structure having properties allowing the passage of chalk is constructed from one panel made of a porous material with moisture-wicking properties and a second panel made of a material with perforations to allow for the passage of chalk from the interior cavity.

10. The apparatus as in claim 1 wherein the exterior back structure having properties preventing the passage of chalk is constructed from one panel made of a non-porous material with moisture-wicking properties and a second panel made of a material with no perforations.

11. The apparatus as in claim 1 wherein the interior cavity structure having properties allowing the retention and distribution of chalk is a void formed between two panels made of a non-porous material to hold chalk and consistently distribute it in all orientations, in particular in a vertically oriented disposition.

12. The apparatus as in claim 1 wherein the interior cavity structure having properties allowing the retention and distribution of chalk is a void formed between two panels made of a porous material to hold chalk and consistently distribute it in all orientations, in particular in a vertically oriented disposition.

13. The apparatus as in claim 1 wherein the sides and the bottom of the apparatus along with the various panels are held together by stitches or other means to form the structure of the apparatus.

14. The apparatus as in claim 1 wherein the opening structure to access the chalk held in the interior cavity of the apparatus is comprised of a slider-operated zipper assembly.

15. The apparatus as in claim 1 wherein the opening structure to access the chalk held in the interior cavity of the apparatus is comprised of a non-zipper related means such as hook and loop tape, snaps, or fold-over type arrangement.

16. The apparatus as in claim 1 wherein the opening structure to access the chalk held in the interior cavity of the apparatus is comprised of reinforcement materials at the top corners to prevent chalk from spilling from the apparatus.

17. The apparatus as in claim 1 wherein the attachment means to secure the apparatus to the body comprise at least one of:
a) straps with fasteners or buckles;
b) straps with hook and loop closure;
c) detachable straps;
d) fixed straps;
e) attachment clips;
f) adhering with adhesive;
g) hook and loop tape; and
h) stitching to clothing.

18. The apparatus as in claim 1 wherein the apparatus can be constructed without attachment means and set on the floor of the gym for use by the athlete in the same manner as the apparatus with the attachment means.

19. The apparatus as in claim 1 wherein the front and the back of the apparatus have re-closable pockets.

20. A method of using an apparatus for carrying and dispensing chalk, the method comprising:
a) providing an apparatus with an exterior front and back surface;
b) providing an apparatus with an interior cavity for holding chalk;
c) filling the interior cavity of the apparatus with chalk;
d) squeezing and massaging the chalk from the exterior front and back surface of the apparatus to evenly distribute the chalk contained in the interior cavity of the apparatus;
e) accessing the chalk from the interior cavity of the apparatus by palming or slapping at least one of, the exterior front and back surface of the apparatus, to get chalk on the palms of the hands to provide friction for gripping a bar, rock or other surface; and
f) pressing the palms of the hands against at least one of, the exterior front and back surfaces of the apparatus, to remove moisture and chalk from the palms of the hands.
